(19) United States
(12) Patent Application Publication
(10) Pub. No.: US 2009/0326285 A1
(43) Pub. Date: Dec. 31, 2009

(54) Use of Supported Mixed Metal Sulfides for Hydrotreating Biorenewable Feeds

(76) Inventors: Lorenz J. Bauer, Des Plaines, IL (US); Edwin P. Boldingh, Des Plaines, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/164,801

(22) Filed: Jun. 30, 2008

(51) Int. Cl. C07C 1/00

(52) U.S. Cl. 585/240

(57) ABSTRACT

Methods for hydroconverting a biorenewable feed with an unsupported sulfided metal catalyst formed by reacting a metal containing compound with a sulfur containing compound to form an insoluble particulate sulfided metal catalyst.

### BACKGROUND OF THE INVENTION

(1) Field of the Invention

This invention concerns methods for the hydroconversion of biorenewable feedstocks using a catalyst systems comprising insoluble dispersed particulate sulfided metal catalyst made by admixing a metal containing compound with a sulfur containing compound.

(2) Description of the Art

The conversion of biorenewable feedstocks to chemicals and fuels that may be further processed in conventional hydrocarbon-based refinery processes requires the biorenewable feedstocks be subjected to both depolymerization and heteroatom removal. One solution known in the art for heteroatom removal is to use either noble metal type catalysts with high activity at low temperatures or conventional supported CoMo, NiMo or NiW metal sulfided catalysts. Typically these catalysts are used in fixed bed reactors.

These known catalyst and reaction systems are problematic because noble metal catalysts are expensive. Moreover, with noble metal catalysts, the high heteroatom hydrogenolysis activity at low temperature is accompanied by high hydrogen uptake. Alternative base metal catalysts, in contrast, have relatively low activities and must operate at high temperatures—where undesirable char formation is favored. Moreover, supported metal catalysts of all types are prone to deactivation by coking. It is also difficult to physically contact solid particulate biorenewable feedstocks with supported metal catalysts. Finally, biorenewable feedstocks include large amounts of oxygen in comparison to petroleum based hydrocarbons. Most supported metal catalysts have been tailored to remove nitrogen and sulfur from petroleum and they have not be tailored or optimized to remove oxygen from biorenewable feedstocks. There is a need therefore, for inexpensive and active catalyst systems that are useful for converting biorenewable feedstocks into low oxygen-containing products that can be used as feeds to conventional refinery processes.

### SUMMARY OF THE INVENTION

This invention includes methods for the hydroconversion of biorenewable feedstocks that employ unsupported particulate metal catalyst systems that are formed by admixing solid, oil soluble, or water soluble metal containing compound with one or more sulfur containing compounds or feedstocks.

One aspect of the invention is a hydroconversion method comprising the steps of: admixing a metal containing compound with a sulfur containing compound to form an insoluble particulate sulfided metal catalyst; combining the particulate sulfided metal catalyst with at least one biorenewable feedstock to form a combined feed; reacting the combined feed in a hydroconversion reaction zone at hydroconversion reaction conditions for a period of time sufficient to form a hydroconversion reaction product; and withdrawing the hydroconversion reaction product from the reaction zone.

In another aspect, this invention non-sulfided metal containing compounds are added to a biorenewable feedstock. The sulfur level in the feedstock is adjusted by sulfur compound addition to provide sufficient sulfur to form insoluble metal sulfides. The non-sulfided metal compounds can be dissolved in water or an organic matrix, or added directly as a solid. The metal compounds are combined sulfur containing compounds and hydrogen at elevated temperature and pressure to form dispersed insoluble metal sulfide particles.

The use of catalyst of this invention in a slurry reactor scheme allows the hydroconversion process to operate under more severe conditions which allows for the higher heteroatom removal and depolymerization and conversion of the biorenewable feedstock into liquids useful as feedstocks for fuel and chemical applications.

### DESCRIPTION OF THE FIGURES

FIG. 1 is a hydroconversion process schematic of this invention.

### DETAILED DESCRIPTION OF THE INVENTION

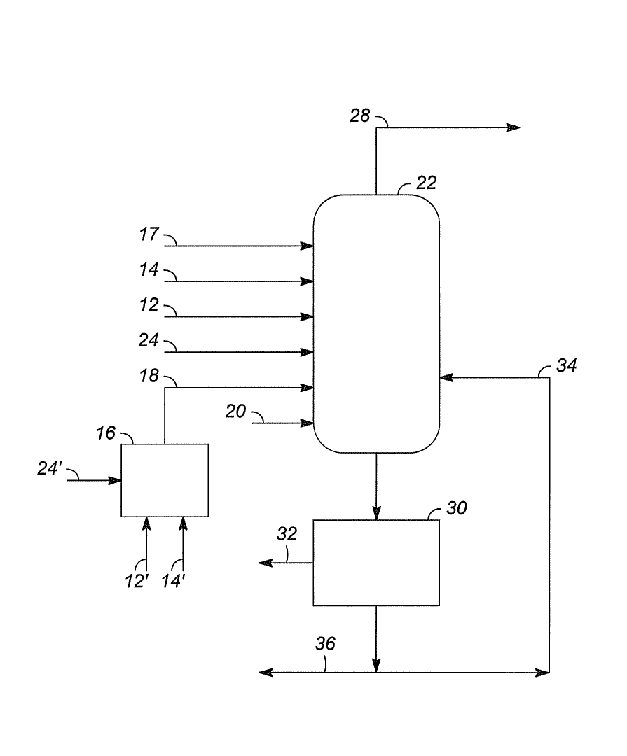

The present invention relates to methods for the hydroconversion of a biorenewable feedstock using an unsupported and insoluble particulate sulfided metal catalyst. The methods of this invention are described below generally with reference to FIG. 1. FIG. 1 is a schematic of one embodiment of a biorenewable feedstock hydroconversion method of this invention. In FIG. 1, biorenewable feedstock stream 12 is directed into hydroconversion reaction zone 22. Other streams directed into hydroconversion reactor 22 include a metal containing compound stream 12, a sulfur containing compound stream 14 and a hydrogen containing gas stream 20. In this embodiment, as will be discussed below, the metal containing compound and the sulfur containing compound combine in hydroconversion reaction zone 22 at hydroconversion reaction conditions to form an insoluble particulate sulfided metal catalyst. In an alternative feed embodiment, also shown in FIG. 1, an insoluble particulate sulfided metal catalyst is formed in a heated vessel 16 by admixing metal containing compound stream 12′ and sulfur containing compound stream 14′ in vessel 16 in the presence of hydrogen and at temperatures of at least about 250° C. and more preferably at least about 300-350° C. to form insoluble particulate sulfided metal catalyst stream 18 and then directing the catalyst stream 18 into hydroconversion reaction zone 22. In yet another embodiment, a combined feed stream may be formed adding a biorenewable feed steam 12′ into vessel 16 or with catalyst stream 18 following vessel 16 to form a combined feed stream and then directing the combined feed stream into hydroconversion reaction zone 22.

In a preferred embodiment, biorenewable feed stream 24, soluble metal containing compound stream 14, sulfur containing compound stream 16 and optional promoter stream 17 are individually directed into hydroconversion reaction zone 22 where the catalyst precursor materials react to form an insoluble particulate sulfided metal catalyst after which the catalyst is available to catalyze the reactions in the hydroconversion reaction zone including depolymerization, heteroatom removal and hydrogenation reactions.

Biorenewable feedstock stream 24 can be a liquid, particulate solid or a combined liquid/particulate solid feed stream. Useful biorenewable feedstocks may include but are not limited to lignin, plant parts, fruits, vegetables, plant processing waste, wood chips, chaff, grain, grasses, corn, corn husks, weeds, aquatic plants, hay, paper, paper products, recycled paper and paper products, and any cellulose containing biological material or material of biological origin.

Lignocellulosic biomass, or cellulosic biomass consists of the three principle biopolymers cellulose, hemicellulose, and lignin. The ratio of these three components varies depending on the biomass source. Cellulosic biomass might also contain lipids, ash, and protein in varying amounts. The economics for converting biomass to fuels or chemicals depend on the ability to produce large amounts of biomass on marginal land, or in a water environment where there are few or no other significantly competing economic uses of that land or water environment. The economics can also depend on the disposal of biomass that would normally be placed in a landfill. Preferred biorenewable feedstocks are liquid phase biorenewable feedstocks including, but not limited to vegetable oils, pyrolysis oils and combinations thereof. The term pyrolysis oil or pyrolytic oil refers to liquid and solid (char) material extracted by destructive distillation from biomass and in particular dried biomass. The destructive distillation occurs in a reactor operating at a temperature of about 500° C. with subsequent cooling. Pyrolytic oil normally contains levels of oxygen that can be as high as 50 wt % (due, in part, to a high water content of from about 8 wt % to about 20 wt % or more) and that are too high for it to be considered a hydrocarbon and, as such, it is distinctly different from similar petroleum products.

Biorenewable feedstocks, such as vegetable oils, pyrolysis oils and lignocellulosic biomass contain organic materials that have a high oxygen content in comparison to petroleum-derived hydrocarbons. Indeed, the biorenewable feeds will typically have an oxygen content of at least 5 wt % and generally at least 20 wt % with a maximum oxygen content of no more that about 50 wt %.

In an alternative embodiment, the hydroconversion feed may include a mixture or conventional hydrocarbon-type hydroconversion feeds and one or more biorenewable feeds.

Where biorenewable feedstock 24 is or includes a solid particulate material, then the particles may be any size that can be processed in the chosen hydroconversion reaction zone. However, it is preferred that a particulate biorenewable feedstock will have a mesh size less than about 50, more preferably less than about 100 mesh and most preferably less than about 200 mesh (75 microns).

In order to improve catalyst dispersion in the hydroconversion reaction zone, the catalyst precursor steams may be combined with a cutting stock and reacted together in vessel 16 to form a catalyst slurry prior to the addition of the catalyst into the reaction zone or otherwise combined with biorenewable feed stream 24. The cutting stock(s) may be any type of material known in the art for creating a catalyst slurry. In the present invention, one useful cutting oil is a pyrolysis oil or any other type of biorenewable oil that is useful in the present invention. In another alternative, the cutting stock may be a hydroconversion reaction zone recycle stream, by product or product stream material. In yet another alternative, the cutting stock may be an inexpensive light oil such as mineral oil. Aqueous solutions of catalyst precursor materials can also be used. Such aqueous precursors are typically added to a feedstock or cutting stock to form an emulsion in which the solid particulate catalyst is formed.

The catalyst used in the methods and processes of this invention are unsupported catalysts. By “unsupported”, it is meant that the catalyst do not include and are not associated with inert support materials such as aluminas, silicas, MgO, carbons and so forth. Instead, the catalysts used in the present invention are microparticulate solid metal catalysts that are prepared from catalyst precursor materials such as water soluble, oil soluble or gaseous precursor materials. When the catalyst precursor materials are admixed in the presence of heat, the precursor materials form very small solid particulate sulfided metal catalyst. The solid particulate sulfided metal catalyst will generally be nanosized or microparticulate particles having an average particle size of less than about 100 microns and preferably about less than about 20 microns. Forming very small insoluble sulfided metal catalyst particles aids in the dispersion of the catalyst throughout the hydroconversion reaction zone improves the contact of the catalyst with the biorenewable feedstock.

The catalyst precursor materials used in the present methods include a metal containing compound, a sulfur containing compound, and an optional promoter. The metal containing compound will generally be an oil soluble or water soluble compound including one or more metals selected from metals such as cobalt, molybdenum, nickel, iron, vanadium, tin, copper, ruthenium and other Group IV-VIII transition metals. More preferably, the metal containing compound is a water soluble or an oil soluble compound including one or more metals selected from the group consisting of molybdenum, cobalt, iron, nickel, ruthenium, tin, copper and combinations thereof. The metal containing compound will be added to the hydroconversion reaction zone in a weight amount that is based upon the weight of the metal in the compound and that is also based upon the biorenewable mass feed rate. Generally the metal containing compound feed rate will range from about 50 ppm to up to 5 wt % of metal based upon the mass feed rate of biorenewable feed to the hydroconversion reaction zone. Preferably the metal rate will range from about 100 ppm to 3 wt % metal. The weight feed rate of metal in the metal containing compound added to the hydroconversion reaction zone will depend largely upon the catalytic activity and activity profile of the resulting sulfided metal catalyst.

Useful oil-soluble metal compounds include compounds produced by the combination of an oxide or a salt of metal selected from group IV through group VIII including transition metal-based catalysts derived from the organic acid salt or metal-organic compounds of vanadium, tungsten, chromium, iron, molybdenum etc. Some examples of useful metal compounds include metal ammonium salts, metal sulfates, metal nitrates, metal chlorides, metal naphthanates, metal oxyhdroxides, metal carbamates, metal dithioates, metal oxides and so forth. For example, molybdenum naphthenate and nickel di-2-ethylhexanoate are useful as metal containing compound catalyst precursors of this invention. Non-limiting examples of other useful oil-dispersed metal compounds include molybdenum dithiocarboxylate, nickel naphthenate, ammonium molybdates, iron naphthenate, molybdenum lithiocarboxylate (MoDTC), molybdenum lithiophosphate (MODTP) as well as their mixtures.

Examples of useful water-soluble dispersed metal containing compounds useful as catalyst precursors of this invention include, but are not limited to sodium molybdate, nickel nitrate, iron nitrate precursors of water-soluble multi-metal composite catalysts, water-soluble ammonium heptamolybdate (AHM), ammonium paramolybdate (APM), and ammonium tetrathiomolybdate (ATM).

One or more metal containing compounds are combined with one or more sulfur containing compounds at high heat to form the insoluble particulate sulfided metal catalysts useful in the methods of this invention. Some examples of useful sulfur containing compounds include but are not limited to hydrogen sulfide gas, organic sulfides such as DMDS, polysulfides, elemental sulfur, sodium sulfide, thiophene, and so forth.

One or more metal containing compounds are generally combined with one or more sulfur containing compounds at molar ratios of metal to sulfur ranging from at least about 1:1.5 to 1:10 or more and preferably from at least 1:2 to about 1:5 or more. The sulfur in the sulfur containing compounds combines with the metals in at a molar ratio of about 2:1 to form the solid sulfided metal catalyst useful in the methods of this invention. Therefore, it is preferred that a molar excess amount of sulfur is combined with the metal containing compound to form the catalysts of this invention.

As noted above, all elevated temperatures are necessary to initiate the formation of insoluble particulate metal sulfide catalysts of this invention from the catalysts precursor materials. Moreover, hydrogen must be present before the solid particulate catalysts can form. Therefore, the location in the hydroconversion process where the insoluble particulate metal sulfided catalyst is formed can be controlled by controlling the point where hydrogen is added into the process. For example, hydrogen can be added to vessel 16 to promote catalyst formation outside of hydroconversion reactor 22 in FIG. 1. Alternatively, the catalyst precursor materials can be combined in the absence of hydrogen and directed into hydroconversion reaction zone 22 where, in the presence of hydrogen, they react to form a well dispersed insoluble particulate metal sulfided catalyst.

The resulting insoluble particulate sulfided metal catalysts of this invention may be used alone or they may be further enhanced by adding small amounts of promoters and/or they may be used along with other well know catalyst additives. In one embodiment, small percentages of at least one active metal such as palladium, platinum, nickel, tungsten, cobalt, nickel, or mixtures thereof are incorporated into the catalysts. It is preferred that a Group IV-Group VIII metal is combined with the catalyst precursors to form a promoted and soluble microparticulate sulfided metal catalyst. More preferably, a promoter metal selected from the group consisting of nickel, cobalt or mixtures thereof are incorporated into the unsupported solid catalyst of this invention. The promoter metal is added to the solid catalyst of this invention in the form of water or oil soluble or insoluble metal compounds. If a promoter metals is used then, it is preferred that the promoter metal compound is the same class of compound as the metal containing compound in order to minimize the number of by product materials in the hydroconversion reaction zone product stream. For example, if the metal containing compound is an ammonium compounds, then it is preferred, but not required that the promoter metal is also an ammonium compound.

The optional promoter metal compound will be combined with the other catalyst precursor materials before the solid catalyst is formed. The promoter metal compound will be added to the other catalyst precursor materials in an amount based upon the weight of metal in the promoter metal compound. Generally the promoter metal will be combined with the other catalyst promoter materials in a weight amount of promoter metal ranging from about 0.5 wt % to about 15 wt % of the weight amount of the metal in the metal containing compound being added to the hydroconversion reactor and more preferably at a weight ranging from about 1 wt % to about 10 wt %.

The solid particulate metal sulfide catalyst is a three dimensional array of atoms. If a promoter is used, then the promoter becomes distributed within the three dimensional catalyst lattice where, because of its proximity to the metal atoms in the metal containing catalysts, it enhances or promotes the ability of the metal atoms to catalyze reactions such as the removal of heteroatoms from biorenewable feeds while suppressing char formation. In addition, the particulate sulfided metal catalyst and the promoted particulate sulfided metal catalysts catalyze the deoxygenation of biorenewable feedstocks. During deoxygenation, the oxygen in the biorenewable feedstock is converted into water and/or carbon dioxide which is easily separated from the remaining hydroconversion reaction products. The resulting deoxygenated, hydroconversion reaction products are able to be used as feedstocks to conventional downstream fuel and petrochemical processes.

The hydroconversion reactor or reaction zone will include an effective amount of catalyst. An effective amount of catalyst is an amount sufficient to convert at least some of the combined feed into lighter hydrocarbon products. The actual effective amount of catalyst that may reside in the hydroconversion reaction zone will vary depending upon the type and activity of the catalyst selected. For example, the amount of catalyst can be as low as about 100 ppm (based upon the weight of the catalyst metal) when a high activity metal such as a cobalt or molybdenum based catalyst is used. It is also possible that the hydroconversion reaction will include up to about 5 weight percent of a low activity metal. For example, a large amount of iron sulfide would likely be needed to be effective in a hydroconversion reaction zone because of its low activity. The ultimate choice of catalyst and the amount used will depend upon one or more factors including, but not limited to cost, activity, and susceptibility to fouling and poisoning and so forth.

Since water is present in the combined feed and/or produced in the hydroconversion reaction zone, additives that bind with water or that control the reaction pH can optionally be added into the reaction zone. Ultimately, any additives known to one skilled in the art as being useful in conjunction with the types of catalysts or the types of process used in the present invention can be added into the reaction zone or combined with the feeds or catalysts introduced into the hydroconversion reaction zone.

A hydrogen containing gas stream 20 is added to the hydroconversion reaction zone to maintain the hydroconversion pressure within the desired range. The hydrogen containing gas stream may be essentially pure hydrogen or it may include additives such as hydrogen sulfide impurity or recycle gasses such as light hydrocarbons. Reactive or non-reactive gases may be combined with hydrogen and introduced into the hydroconversion reaction zone to maintain the reaction zone at the desired pressure and to achieve the desired hydroconversion reaction product yields.

The hydroconversion reaction zone of this invention may be selected from any type of hydroconversion reactor that is useful for converting low value heavy hydrocarbons into high value lighter hydrocarbons. The hydroconversion reaction zone may include two or more reactors operating at different reaction severities or it may be a single reactor. A single reactor is preferred in the present methods as the inventors have surprisingly found that their catalyst is able to deoxygenate and depolymerize the biorenewable feed without significant char formation in a single reactor.

The hydroconversion reaction zone will include a dynamic catalyst bed. Some non-limiting examples of useful dynamic catalysts bed reaction systems useful in the present invention include, but are not limited to, the VEBA-combi-cracking process, M-coke technology as disclosed in U.S. Pat. No. 4,134,825 B1, the CANMET process which is disclosed for example in U.S. Pat. No. 4,299,685 B1, the SOC technology which uses highly dispersed super fine powder of transition metallic compounds at high reaction presses, the (HC)₃ process such as disclosed in U.S. Pat. No. 5,578,197 B1 and homogeneous catalysts hydroconversion reaction processes and methods such as those disclosed in U.S. Patent Application No. 2005/241993.

The combined feeds and catalysts of this invention may also be combined and hydroconverted in the processes and apparatuses described in U.S. Pat. No. 6,517,706 B1, the specification of which is incorporated herein by reference. The '706 patent discloses processes for converting a slurry feed of a heavy hydrocarbon feedstock and coke-inhibiting additive particles together with a hydrogen-containing gas. The slurried feed ingredients are fed upward through a confined hydrocracking zone in a vertical, elongated, cylindrical vessel with a generally dome-shaped bottom head. A mixed effluent is removed from the top containing hydrogen and vaporous hydrocarbons and liquid heavy hydrocarbons. The slurry feed mixture and a portion of the hydrogen-containing gas are fed into the hydrocracking zone through an injector at the bottom of the dome-shaped bottom head and the balance of the hydrogen-containing gas is fed into the hydrocracking zone through injection nozzles arranged within of the hydrocracking zone at a location above the slurry-feed injector. The combined slurry feed and hydrogen-containing gas are injected at a velocity whereby the additive particles are maintained in suspension throughout the vessel and coking reactions are prevented.

The hydroconversion reaction will take place at hydroconversion reaction conditions sufficient to obtain the desired light hydro carbon yield from the combined feed. The reaction conditions will generally include temperatures ranging from 300 to 600° C. More preferably from 350 to 500° C. and most preferably 425 to 500° C. The useful hydroconversion reaction pressures will typically range from about 1000 to about 3000 psig and more preferably from about 1200 to about 2500 psig. At these conditions, the biorenewable feeds are deoxygenated and cracked to form lower boiling materials that are useful as feedstocks to fuel and petrochemical processes.

Referring again to FIG. 1, the hydroconversion reactor/reaction zone 22 will generally include a gaseous product stream 28 and a slurry product stream 26. Slurry product stream 26 will generally be directed into a device 30 that effectively separates at least some of the solid material in the slurry from the liquid material. Device 30 may be a filter, slurry separators, centrifuges, distillation to remove the solids such as pitch, or any other device or apparatus used in hydrocarbon processing for separating or concentrating solids in a solids containing liquid stream. A liquid product stream 32 will be removed from hydroconversion reactor 22 and further processed in down stream processes to concentrate and recover high value hydrocarbons from the liquid product stream 32. In most cases, the liquid product stream will be used as is or will be separated and the separated components used as feed stocks for traditional refinery processes. Off gas 28, which may also contain high value light hydrocarbons will also be processed in traditional refinery processes to convert and/or recover high value materials such as light hydrocarbons, hydrogen and so forth. Both product streams 28 and 32 can also be processed in down stream processes to remove unwanted contaminants such as water, sulfur, oxygen, and so forth from the streams.

Device 30 also forms a concentrated slurry stream that can include solid catalyst and that will include solid biorenewable feedstock that was not converted into a liquid or gaseous product in the hydroconversion reactor. A portion of the concentrated slurry stream formed device 30, possibly containing solid catalyst, can be a recycle stream 34 that is directed back into hydroconversion reactor 22. In addition, an amount of the concentrated slurry formed in device 30 ranging from a slip stream to all of the concentrated slurry can be removed from the process via 36 for separation, pitch removal processing and/or disposal.

### EXAMPLE

In this hypothetical example, ammonium molybdate heptahydrate is dissolved in between 2 to 10 times its weight of water and optionally combined with a group VIII promoter metal such as nickel and cobalt. The molar ratio of Ni or Co to the molybdenum can range from 1:100 to about 1:2. The group VII metal can also be added at as a sulfate. The pH aqueous of the solution should be adjusted to between 8-10 by addition of ammonia. The aqueous solution should be vigorously mixed with about 2-50 times its weight of a biorenewable feedstock, such as vegetable oil or pyrolysis oil and the admixture reacted in a batch reactor at a pressure of about 500-2500 psig, with a preferred pressure of 1500-2000 psig, and a temperature of about 350-460° C., with a preferred temperature of about 420° C. and with the addition of hydrogen at 60 NL per Kg of feed. The goal is to supply amount of catalyst to the reactor sufficient for the weight of the non-promoter metal in the catalyst slurry to range from between 1-10% of weight of the bulk feed. If the biofeed stock is a solid, a cutting fluid can be used to form a dispersed catalyst before it reacts with the biofeed. Preferably the cutting oil is a low value recycle stream from the products of the reaction. A sulfur containing compound or sulfiding material is also added to the reactor to form the solid metal catalysts. If H₂S is used as the sulfiding material, it can be added along with hydrogen to the slurry mixture as it is pumped to the slurry reactor. If elemental sulfur or an organic sulfide like dimethysulfide is used, it is added to the slurry during the mixing step. The amount of sulfur added should be between 1.5 to 3.5 times the Mo plus group VII metal on a molar basis. The hydrogen is added to the catalyst slurry and the combined material is combined with the bulk of the feed in the slurry reactor. The amount of Mo in the reactor can between 50 ppm and 1 wt %, with a preferred level of 1000 ppm

This invention has been discussed generally with reference to the drawing. The drawing depicts particular embodiments of the invention and are not intended to limit the generally broad scope of the invention as set forth in the claims. Moreover the specifications of U.S. Pat. Nos. 4,134,825, 4,299,685, 5,578,197, 6,517,706 and U.S. Patent Application No. 2005/0241993 which are discussed above are incorporated herein by reference.

1. A hydroconversion method comprising the steps of:
a. admixing a metal containing compound with a sulfur containing compound to form an insoluble particulate sulfided metal catalyst;
b. combining the insoluble particulate sulfided metal catalyst with at least one biorenewable feedstock to form a combined feed;
c. reacting the combined feed in a hydroconversion reaction zone at hydroconversion reaction conditions for a period of time sufficient to form a hydroconversion reaction product; and
d. withdrawing the hydroconversion reaction product from the reaction zone.

2. The method of claim 1 wherein the metal in the metal containing compound is a Group IV-VIII transition metal.

3. The method of claim 1 wherein the metal in the metal containing compound is selected from the group Mo, Co, Fe, Ni, Ru, Sn, Cu, and combinations thereof.

4. The method of claim 1 wherein the metal containing compound is an oil soluble or water soluble metal compound.

5. The method of claim 1 wherein the metal containing compound is an oil soluble or water soluble molybdenum compound.

6. The method of claim 1 wherein the metal containing compound is a metal oxide powder.

7. The method of claim 1 wherein the biorenewable feedstock is a liquid biorenewable feedstock, a solid particulate biorenewable feedstock or combinations thereof.

8. The method of claim 1 wherein the combined feed includes a hydrocarbon feed component.

9. The method of claim 1 wherein the combined feed has a total oxygen content of at least 5 wt % and no more than 50 wt %.

10. The method of claim 1 wherein a promoter is added to the sulfided metal catalyst including a promoter.

11. The method of claim 10 wherein the promoter is at least one Group IV-Group VIII transition metal and wherein the promoter metal is present in the sulfided metal catalyst in an amount ranging from about 0.5 wt % to about 15 wt % of the weight of metal in the sulfided metal catalyst.

12. The method of claim 11 wherein the promoter is selected from Ni, Co, and mixtures thereof.

13. The method of claim 1 wherein the sulfur compound is combined with the metal compound to form an insoluble particulate metal sulfided catalyst having a molar ration of sulfur to metal ranging from about 1.5 to about 2.

14. The method of claim 1 wherein the insoluble particulate sulfided metal catalyst accumulates in the hydroconversion zone and then is withdrawn in an amount sufficient to maintain an amount of insoluble particulate sulfided metal catalyst in the reactor ranging from about 100 ppm to about 5 weight percent.

15. The method of claim 1 wherein the biorenewable feedstock includes a liquid biorenewable feedstock selected from one or more pyrolysis oils, one or more vegetable oils and combinations thereof.

16. The method of claim 1 wherein admixing step (a) is performed before the insoluble particulate sulfided metal catalyst is directed into the hydroconversion reaction zone.

17. The method of claim 1 wherein admixing step (a) is performed in the hydroconversion reaction zone.

18. The method of claim 1 wherein the hydroconversion reaction zone is a single dynamic bed hydroconversion reactor.
